(19) United States
(12) Patent Application Publication
(10) Pub. No.: US 2011/0004121 A1
(43) Pub. Date: Jan. 6, 2011

(54) BIOPSY DEVICE HANDLE

(75) Inventors: Lev Drubetsky, Coquitlam (CA); Yakov (Jacob) Gofman, Richmond (CA)

Correspondence Address: IPHORGAN, LTD., 1130 LAKE COOK ROAD, SUITE 240, BUFFALO GROVE, IL, 60089, US

(73) Assignee: ANGIOTECH PHARMACEUTICALS INC., Vancouver (CA)

(21) Appl. No.: 12/790,762

(22) Filed: May 28, 2010

Related U.S. Application Data
(60) Provisional application No. 61/182,006, filed on May 28, 2009.

(51) Int. Cl. A61B 10/02

(52) U.S. Cl. 600/567

(57) ABSTRACT

A biopsy device handle for use with a biopsy device needle set, comprising a housing, a throw depth selection assembly, a cocking assembly, a carriage assembly, a needle set driver assembly, and an actuating assembly. A safety feature may be incorporated into the device to reduce or eliminate the chance of unintentional firing of the biopsy device.

### CROSS REFERENCE TO RELATED APPLICATIONS

This application claims the benefit under 35 U.S.C. §119(e) of U.S. Provisional Patent Application No. 61/182,006, filed May 28, 2009, which application is incorporated herein by reference in its entirety.

### FIELD OF INVENTION

The present invention relates generally to handles for biopsy devices.

### BACKGROUND OF THE INVENTION

It is often desirable and frequently absolutely necessary to sample or test a portion of tissue from humans and even animals to aid in the diagnosis and treatment of patients with cancerous tumors, pre-malignant conditions and other diseases or disorders. Typically in the case of cancer or the suspicion of malignant tumors, a very important process called tissue biopsy is performed to establish whether cells are cancerous.

Biopsy may be done by an open or closed technique. Open biopsy removes the entire tissue mass or a part of the tissue mass. Closed biopsy on the other hand is usually performed with a needle-like instrument and may be either an aspiration (hollow needle on a syringe) or a core biopsy (special tissue cutting needle design). In needle aspiration biopsy, individual cells or clusters of cells are obtained for cytologic examination. In core biopsy, a segment of tissue is obtained for histologic examination which may be done as a frozen section or paraffin section.

The methods and procedures of obtaining tissue samples for cytologic or histologic examination have been performed historically by manual insertion and manipulation of the needle. These procedures are performed “blind” by the physician and guided by “feel” and known anatomic “landmarks”.

Tumors are first noted in a patient by one of three ways, palpation, x-ray imaging or ultrasound imaging. Once a tumor is identified, a biopsy procedure is performed. Modern medical opinion dictates early detection of cancer, which increases the likelihood of successful treatment. Biopsies are performed on “Tumor Masses” as small as 2 millimeters in diameter. This procedure is performed under ultrasound or x-ray guidance. Tumors of this size cannot be biopsied reliably by hand since the tumor is about the same size as the biopsy needle. Manual attempts at biopsy can push the tumor away without piercing the mass. Automatic puncture devices are needed to accelerate the needle at such a velocity that even a small tumor can be pierced.

Two very important innovations in the field of medical technology have influenced the field of tissue biopsy in the last five years. One, the use of tissue imaging devices which allow the physician to “see” inside the body and visually guide the needle to the tumor mass. Two, the invention of the Automatic Core Biopsy Device (ACBD) or “Biopsy Gun”. The ACBD is an instrument which propels a needle set with considerable force and speed to pierce the tumor mass and collect the tissue sample. This ACBD device has allowed physicians to test tissue masses in the early stages of growth and has contributed to the medical trend of early diagnosis and successful treatment of cancer.

Examples of such ACBD devices have been described with respect to the collection of tissue samples in U.S. Pat. Nos. 4,651,752, 4,702,260, and 4,243,048.

### BRIEF SUMMARY OF THE INVENTION

There is provided a biopsy device handle for use with a biopsy device needle set, including a housing, a throw depth selection assembly, a cocking assembly, a carriage assembly, a needle set driver assembly, and an actuating assembly. In certain embodiments, the biopsy device handle further includes a stylet assembly.

In certain embodiments, the needle set driver assembly includes a plurality of cannula drivers for drivably mounting needle set cannulae. In certain of these embodiments, at least one cannula driver is rotatable relative to the housing.

In certain embodiments, the biopsy device handle permits an operator of the device to fully cock the device one-handedly.

These and other aspects of the present invention will become evident upon reference to the following detailed description and attached drawings. In addition, various references are set forth herein which describe in more detail certain procedures and/or compositions (e.g.; devices and their components), and these references are incorporated herein by reference in their entirety.

### DESCRIPTION OF THE DRAWINGS

FIG. 1 is a cross-sectional perspective view seen from above a biopsy device handle in accordance with an embodiment of the invention.

FIG. 2 is a cross-sectional perspective view of a biopsy device handle when the device is in a cocked position in accordance with an embodiment of the invention shown in FIG. 1.

FIG. 3 is a cross-sectional perspective view from above of a biopsy device handle when the device is in a fired position in accordance with an embodiment of the invention shown in FIG. 1.

FIG. 4 is a cross-sectional view from above of a biopsy device handle when the device is in a fired position in accordance with an embodiment of the invention shown in FIG. 1.

FIG. 5 is a cross-sectional view from above of a biopsy device handle after a first cocking stroke in accordance with an embodiment of the invention shown in FIG. 1.

FIG. 6 is a cross-sectional view from above of a biopsy device handle after a second cocking stroke in accordance with an embodiment of the invention shown in FIG. 1.

FIG. 7 is an exploded view of a biopsy device handle in accordance with an embodiment of the invention shown in FIG. 1.

FIG. 8 is a perspective view of a biopsy device handle in accordance with another embodiment of the invention.

FIG. 9 is a cross-sectional perspective view of a biopsy device handle in accordance with an embodiment of the invention shown in FIG. 8.

FIG. 10 is a cross-sectional view from above of a biopsy device handle after a second cocking stroke in accordance with an embodiment of the invention shown in FIG. 8.

FIG. 11 is an exploded view of a biopsy device handle in accordance with an embodiment of the invention shown in FIG. 8.

FIG. 12 is a perspective exterior view of a biopsy device handle in accordance with an embodiment of the invention.

FIG. 13 is a perspective exterior view of a biopsy device handle in accordance with an embodiment of the invention shown in FIG. 12.

FIG. 14 is a perspective exterior view of a biopsy device handle in accordance with an embodiment of the invention shown in FIG. 12.

FIG. 15 is a perspective exterior view of a biopsy device handle in accordance with an embodiment of the invention.

FIG. 16 is an exploded view of a biopsy device handle in accordance with an embodiment of the present invention.

FIG. 17 is an expanded view of an actuator present within a biopsy device handle in accordance with one embodiment of the present invention.

FIG. 18 is a cross sectional perspective view illustrating a needle set driver guide engaged against an actuator, to prohibit unintended firing of one embodiment of the biopsy device of the present invention.

FIG. 19 is a perspective view illustrating various positions for the throw depth stop of the needle set driver guide as located within the biopsy device handle in one embodiment of the present invention.

FIG. 20 is a cross sectional perspective view illustrating an embodiment of the present invention wherein a needle set driver guide is not engaged against an actuator, thereby allowing the biopsy device to be fired.

### DESCRIPTION OF THE INVENTION

Indications of lateral direction such as ‘from above’, ‘upper’, ‘lower’ are defined by the views of the Figures. The terms “proximal” and “distal” refer to the person from whom a biopsy sample is extracted; the terms “front” and “rear” have a corresponding meaning. Thus, the proximal end of a biopsy apparatus is its front end, pointing to the patient.

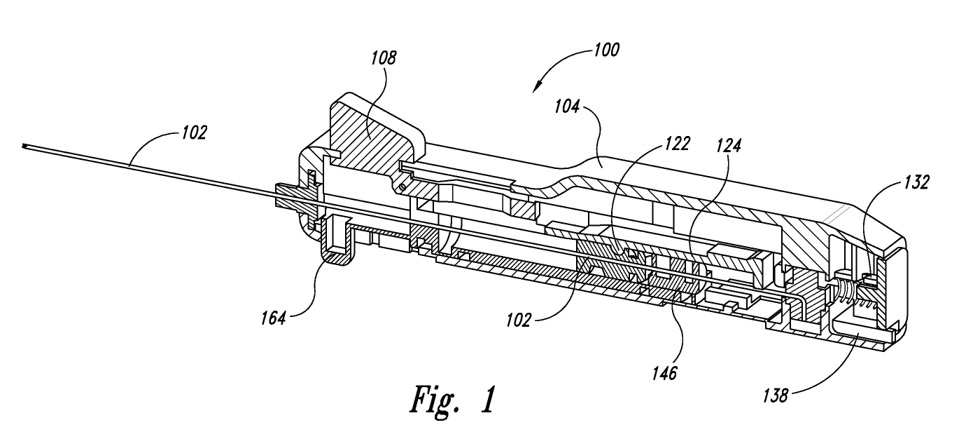

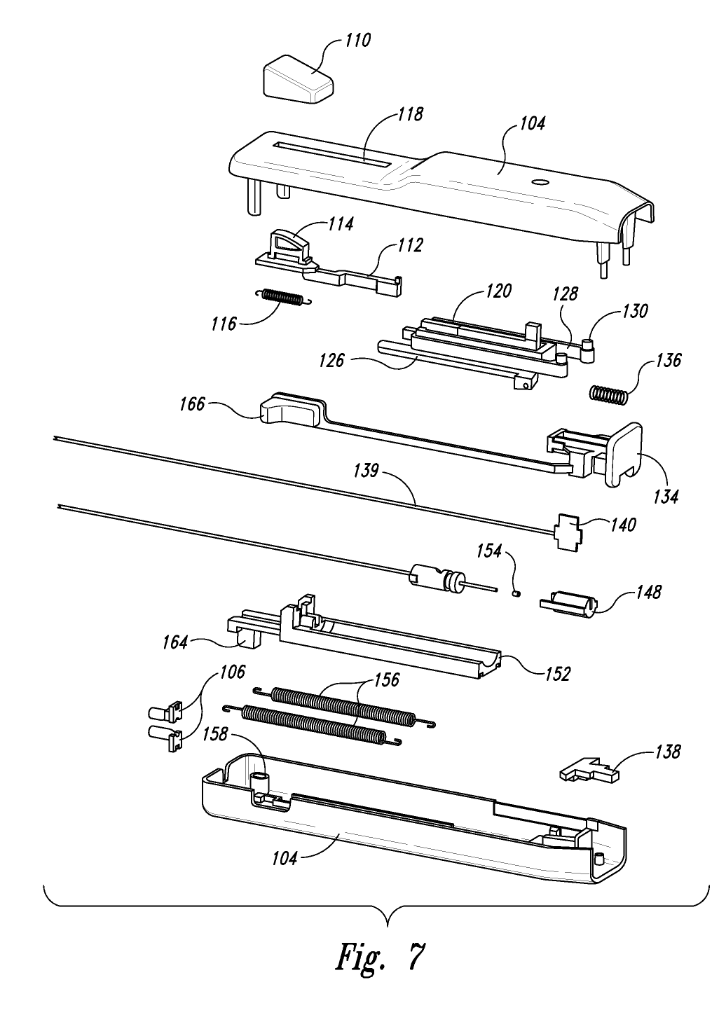

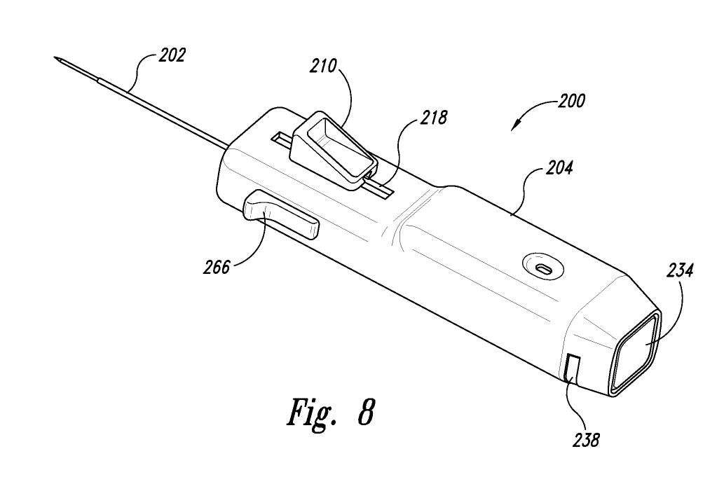

One embodiment of a biopsy device handle is illustrated in FIGS. 1 to 7 with a needle set including a cannula set (which may include one or more cannulae) extending through the proximal end of the handle. The handle of this embodiment is provided with a mechanism allowing two cocking strokes to fully cock the device, such that the operator of the device may operate it with one hand. In this embodiment, the length of each cocking stroke is selected to correspond to the motion that an operator's digit (on the same hand used the operator to hold the device) may comfortably impart to it, and the sum of the lengths of the cocking strokes is equal to or greater than, depending on the throw depth and whether or not a twisting motion is to be imparted to all or part of the needle set, the distance traveled by the needle set from a fully cocked position to a fully fired position. In embodiments wherein the distance traveled by a biopsy needle set is greater (for example, where throw depths are greater), it may be desirable to provide additional cocking strokes. In yet other embodiments, only one cocking stroke may be required to fully cock the device, whether the operator uses two hands or one. For example, in embodiments for which it is desired to have two-handed operation of a biopsy device, such that one hand of an operator is used to hold the device while the other is used to cock it, a single cocking stroke that is equal or greater in length than the distance traveled by a needle set from full cocking of the device to full firing could be used. In other embodiments, a mechanism allowing for full single-handed cocking of a biopsy device may be provided with a gear system or the like.

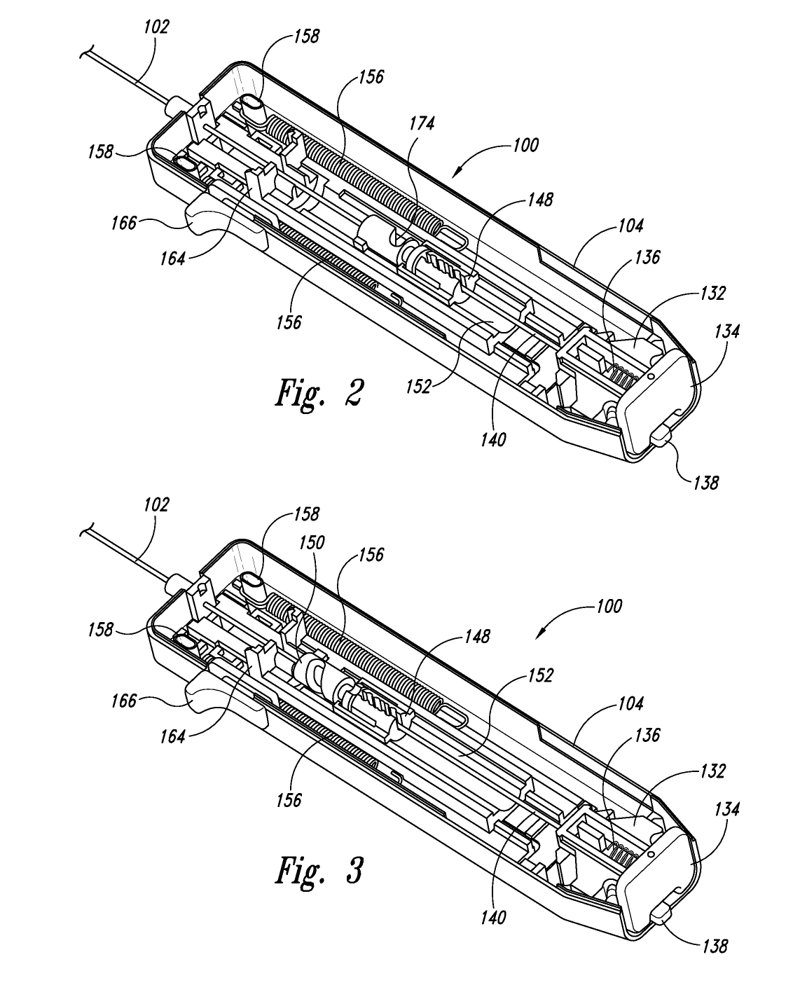

Referring now to FIG. 1, the biopsy device handle 100 includes a housing 104, a cocking assembly 108, a carriage assembly 124, an actuator assembly 132, needle set driver assembly 146, and a throw depth stop 164. Referring additionally to FIGS. 2 to 7, cocking actuator 114 of cocking assembly 108 extends out of housing 104 through aperture 118, and may additionally be provided with cocking actuator button 110. Cocking lever 112 of cocking assembly 108 engages one of a plurality of catches 120 of carriage assembly 124. Carriage drive assembly 156, represented in this embodiment as a pair of tension springs affixed at the other of their ends to mounts 158 near the proximal end of the housing 104, engages carriage 126 of assembly 124, which includes carriage arms 128 that, during the first cocking stroke, slidingly engage intermediate catch guides 160 on housing 104. Carriage 126 also engages outer cannula driver 150, to which outer cannula 144 is drivably mounted, and outer cannula driver 150 in term engages inner cannula driver 148, to which inner cannula 142 is drivably mounted. In certain other embodiments, the carriage may engage another cannula driver, or all such drivers. Inner and outer cannulae are annularly arranged around a stylet. In the present embodiment, stylet assembly 140 includes a stylet with a stylet mount at one end thereof, disposed distally inside housing 104. (In various other embodiments, the needle set may also include a stylet, thereby obviating the need to provide one in the device handle.) Inner cannula 142 and outer cannula 144 extend through an opening in the proximal end of the housing 104, which may be reinforced with a needle set guide 106 (which needle set guide may be integral part of the housing 104 or mounted thereon) to further stabilize guidance of needle set 102.

Inner cannula driver 148 and outer cannula driver 150 are disposed laterally in series and are coupled by cannula driver coupler 154, thus comprising needle set driver assembly 146; as such, displacement of one cannula driver causes displacement of the other. Both drivers are laterally displaceable along needle set driver guide 152. Needle set driver guide 152 is mounted on an inner surface of housing 104 and includes at its proximal end a barrier, comprising part of throw depth stop 164, which determines the travel distance of outer cannula driver 150 and thereby prevents needle set 102 from traveling past the preselected throw depth.

In the present embodiment, the device is cocked in two strokes, the first of which displaces the carriage from its initial uncocked position laterally through the housing to an intermediate position. Referring to FIG. 5, as the device is being cocked during such a first cocking stroke, cocking assembly 108 is displaced distally as the cocking assembly is actuated by the operator applying force to cocking actuator button 110 covering cocking actuator 114. As cocking assembly 108 moves distally, cocking lever 112 engages one of the distal carriage catches 120 and thereby moves carriage assembly 124 along with it. Resilient carriage arms 128 and carriage arm ends 130 are thus displaced along intermediate catch guides 160 (which, in the present embodiment, are located on the inner surface of housing 104). As intermediate catch guides 160 do not, in the present embodiment, run parallel to a central axis of the handle 100 but rather splay out at their distal ends towards the sides of the handle, carriage arms 128 are deformed slightly to splay out towards the sides of the handle as they travel distally along intermediate catch guides 160 during the first cocking stroke. Thus, when carriage arms 128 reach the distal ends of intermediate catch guides 160, carriage arms 128 tend to resile back towards the centre of the handle, to the extent that the continued engagement of carriage arms 128 with intermediate catch guides 160 permit. Once carriage arm ends 130 have moved back towards the center of the handle, the engagement of carriage arm ends 130 with the intermediate catches 162 at the distal end of the intermediate catch guides 160 prevent carriage arms 128 from sliding proximally while carriage drive assembly 156 prevents carriage arms 128 from sliding distally without the application of additional force to displace ends 130 from intermediate catches 162 (as would be provided, for example, with the force of a second cocking stroke). At the same time, referring now to FIG. 2, inner cannula driver 148 and outer cannula driver 150 may travel along needle set driver guide 152 until carriage arm ends 130 reach the intermediate catches 162. This is the intermediate stop, or partially cocked, position.

Once the intermediate stop position is reached and carriage arm ends 130 are engaged in intermediate catches 162, and no more force is applied by the operator to complete the first cocking, cocking repositioner 116 (which, in the present embodiment, is represented by a tension spring attached at one end to the cocking assembly and at its other end to a proximal location on the inner surface of housing 104) urges cocking assembly 108 back to its original, uncocked position.

As, at the end of the first cocking stroke, carriage 126 is displaced distally relative to cocking assembly 108, cocking lever 112 engages one of the proximal cocking catches 120 of carriage assembly 124. As force of the second cocking stroke is applied, cocking lever 112 is again displaced distally and with it displaces carriage 126 further distally, causing the release of carriage arm ends 130 from intermediate catches 162. This time, resilient carriage arms 128 and carriage arm ends 130 are displaced along main catch guides 170 (which, like intermediate catch guides 160, angle away from the central axis of the device handle 100), deforming carriage arms 128 out towards the sides of the handle as they travel distally along main catch guides 170. Carriage arms 128 are thus advanced towards main catches 172, at which carriage arms 128 tend to resile back towards the central axis of the handle, and arm ends 130 are caught in catches 172. Carriage 126 is prevented from moving further distally by tension in the carriage drive assembly 156 and proximally by engagement of arm ends 130 in main catches 172. Displacement of carriage 126 during the second cocking stroke results in the displacement of both outer cannula driver 150 and inner cannula driver 148 further distally along needle set driver guide 152 until carriage arm ends 130 come to rest in main catches 172. This is the fully cocked position; once it is reached and the operator is no longer applying a cocking force, cocking repositioner 116 again returns cocking assembly 108 to its initial position.

As the depth in tissue to which the needle set is to travel may be set using the throw depth selector 166, the position of throw depth selector 166 and throw depth stop 164 limits the distance which inner cannula driver 148 and outer cannula driver 150, and their respective cannulae, may travel proximally. Throw depth stop 164 may be configured to provide any number of throw depths within a given range (which range is determined by the length of the housing opening along which the throw depth selector 166 may be displaced and set), or it may be configured to provide a limited number of throw depths. Throw depth stop 164 is associated with needle set driver guide 152, and displacement of throw depth stop 164 results in displacement of needle set driver guide 152 in housing 104, thus determining how far carriage 126, and therefore inner cannula driver 148 and outer cannula driver 150, may travel within housing 104 to throw depth stop 164 at the proximal end of guide 152.

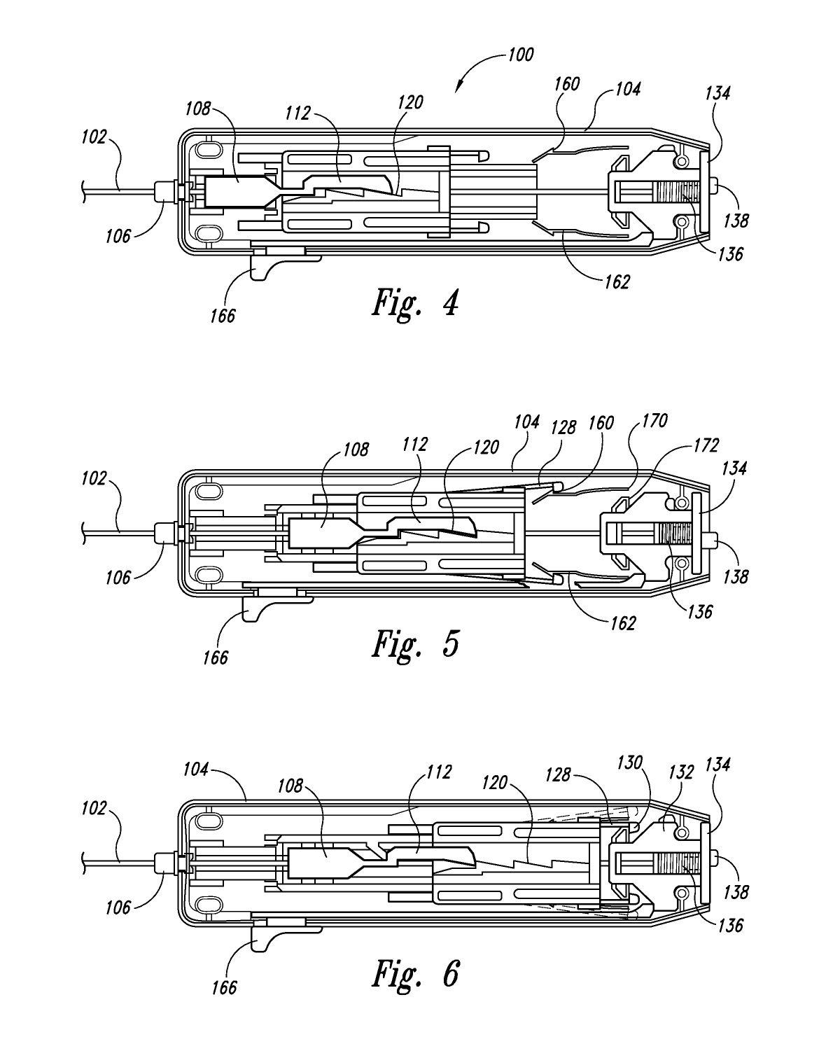

Once the throw depth selected and the device is fully cocked, the needle set may be injected for tissue sampling at a tissue entry site that the device operator has selected. To actuate the device, the operator moves actuator safety 138 below actuator assembly 132 (which, in the present embodiment, also includes actuator 134 and actuator repositioner 136) from its blocking, or “safe”, position which prevents actuating the device to its actuating position centrally below actuator 134 (as illustrated in FIGS. 4 and 5) and then presses actuator 134, urging carriage arm ends 130 out from main catches 172. After releasing carriage arm ends 130 from main catches 172, actuator 134 is returned to its unactuated position by actuator repositioner 136. The operator's pressing action causes actuator 134 to be displaced proximally towards the carriage arm ends 130 in the main catches 172, and forces the carriage arm ends 130 out of the main catches 172. This release of the carriage arm ends 130 allows the release of potential energy in carriage drive assembly 156 (originally stored by the two-stage cocking of the device), which causes carriage assembly 124, and therefore inner cannula driver 148 and outer cannula driver 150, to travel proximally until outer cannula driver 150 reaches the proximal end of needle set driver guide 152. Accordingly, the proximal ends of inner cannula 142 and outer cannula 144 travel away from the device and towards the tissue sampling site.

For embodiments in which a twisting motion is to be imparted to at least one of the cannulae, the corresponding cannula driver rotates during actuation of the device. For cylindrical cannula drivers, the driver may, by way of example, be part of a worm drive (wherein the driver includes a worm on its outer surface and the needle set driver guide or carriage assembly includes a worm gear, or vice versa) or, inter alia, be provided with a spiral groove and pin assembly. It is to be understood that various types of rotation-imparting means may be suitably used, the selection and configuration of which may depend on factors such as manufacturing costs, the degree of rotation desired, and the needle set travel distance over which such rotation is to be imparted.

In the embodiment illustrated in FIGS. 1 to 7, outer cannula driver 150 includes a helical groove 174 on its outer surface which slidingly engages pin 122 (shown in FIG. 1) of carriage assembly 124. Thus, when the proximal end of outer cannula driver 144 reaches the distal end of throw depth stop 164 and is prevented from travelling further proximally, carriage assembly 124 continues to move proximally for a distance no greater than the length of helical groove 174 and so causes outer cannula driver 144 to rotate in order to maintain engagement with pin 122. In various other embodiments, the helical groove might be provided on a carriage assembly and the pin might be provided on the surface of the cannula driver corresponding to the cannula which is to be twisted. In yet other embodiments, one of the helical groove and pin may be disposed on the cannula driver of interest, while the other of the pair may be disposed on a throw depth stop or needle set driver guide.

Once the device has been actuated and injected into tissue, and a biopsy sample captured by the needle set, the needle set may be withdrawn from the tissue site. To retrieve the biopsy sample from the needle set, the device may be cocked again to draw the needle set back. In the embodiment shown in FIGS. 1 to 7, cocking the device after actuation causes the rotation of outer cannula driver 150 in a direction opposite to that which was caused by actuation, and urges both inner and outer cannula drivers 148 and 150 distally. As inner and outer cannulae 142 and 144 are nestedly arranged over the stylet portion of assembly 140 and as stylet assembly 140 remains stationary, the distal movement of cannula drivers 148 and 150 causes cannulae 142 and 144 to travel distally and the proximal end of stylet assembly 140 to inhibit the corresponding distal movement of the biopsy sample. In this fashion, the distal movement of cannulae 142 and 144 exposes and releases the biopsy sample.

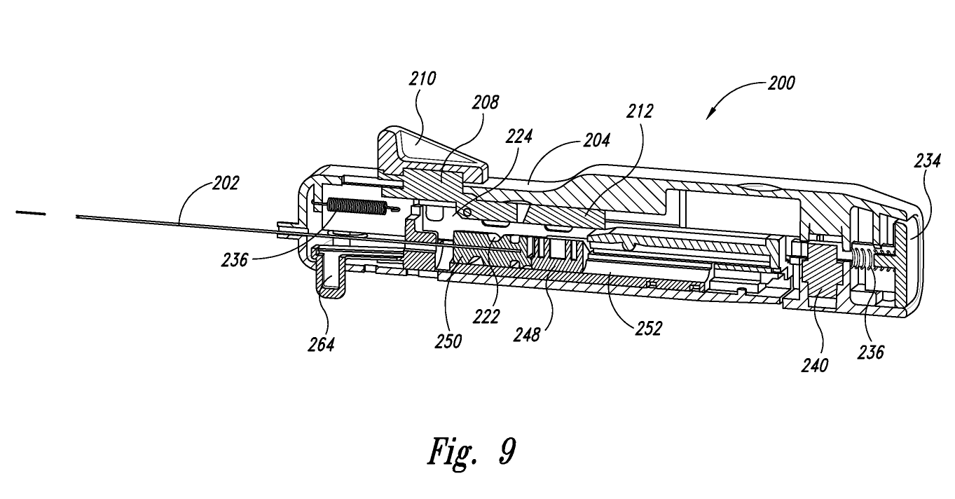

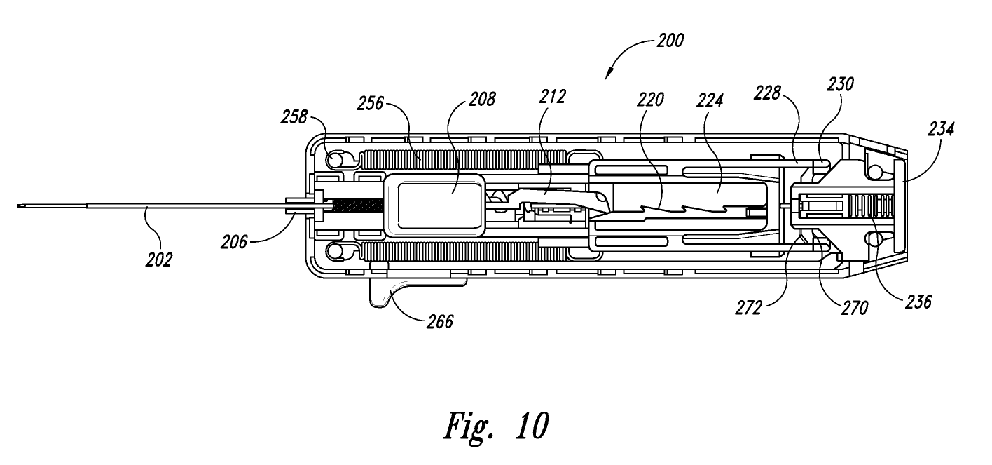

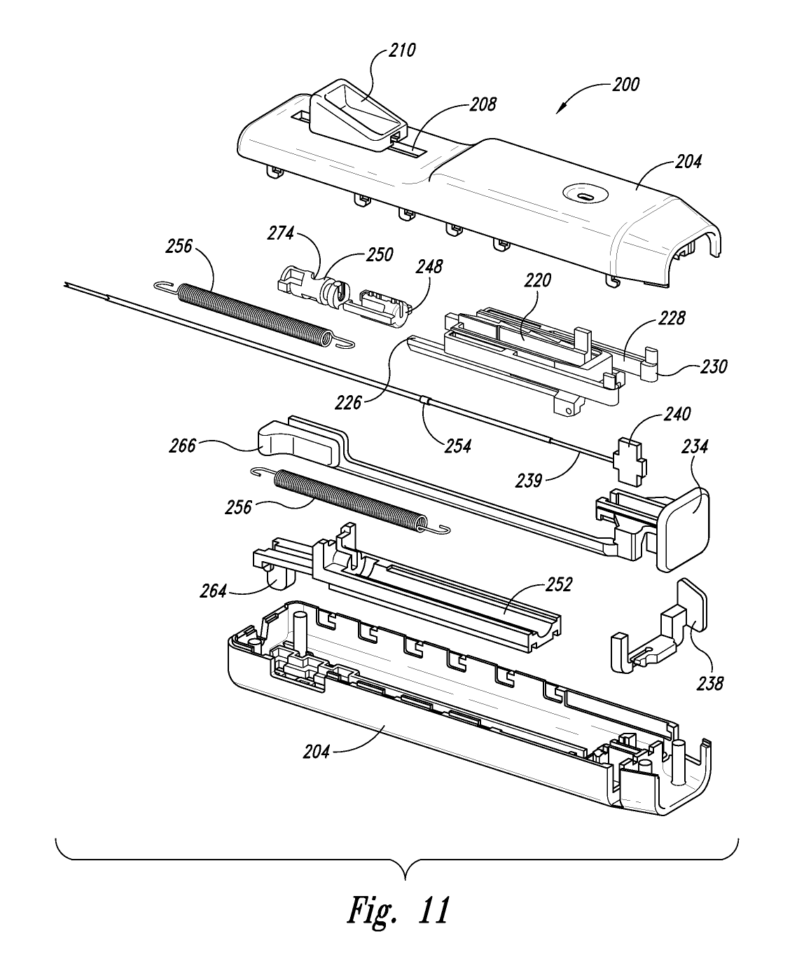

Referring now to the embodiment shown in FIGS. 8 to 11, biopsy device handle 200 as shown in FIG. 1 includes cocking button 210 slidably disposed in aperture 218 and throw depth selector 266 near the proximal end of housing 204, actuator 234 disposed at the distal end of housing 204, and actuator safety 236 disposed near the distal end of housing 204. Needle set 202 is mounted in device handle 200. Referring additionally to FIGS. 9 to 11, the device is cocked in two strokes, the first of which displaces carriage 226 from its initial uncocked position laterally through housing 204 to an intermediate position. Cocking assembly 208 is displaced distally as it is actuated by the operator applying force to cocking actuator button 210. As cocking assembly 208 moves distally, cocking lever 212 engages one of the distal carriage catches 220 and thereby moves carriage assembly 224 along with it. Resilient carriage arms 228 and carriage arm ends 230 are thus displaced to an intermediate (or partly cocked) position. At the same time, inner cannula driver 248 and outer cannula driver 250, coupled by cannula driver coupler 254, travel along needle set driver guide 252 until carriage arm ends 230 reach the intermediate position. Cocking repositioner 216 (which, in the present embodiment, is represented by a tension spring attached at one end to the cocking assembly and at its other end to a proximal location on the inner surface of housing 204) then urges cocking assembly 208 back to its original, uncocked position.

As, at the end of the first cocking stroke, carriage 226 is displaced distally relative to cocking assembly 208, cocking lever 212 engages one of the proximal cocking catches 220. As force from the second cocking stroke is applied, cocking lever 212 is again displaced distally and with it displaces carriage 226 further distally, along main catch guides 270 until carriage arm ends 230 are caught in main catches 272. Carriage 226 is prevented from moving further distally by tension in the carriage drive assembly 256 and proximally by engagement of arm ends 230 in main catches 272. Displacement of carriage 226 during the second cocking stroke results in the displacement of both outer cannula driver 250 and inner cannula driver 248 further distally along needle set driver guide 252 until carriage arm ends 230 come to rest in main catches 272. This is the fully cocked position; once it is reached and the operator is no longer applying a cocking force, cocking repositioner 216 again returns cocking assembly 208 to its initial position.

As throw depth stop 264 is associated with needle set driver guide 252, and displacement of throw depth stop 264 results in displacement of needle set driver guide 252 in housing 204, the operator may set throw depth stop to the desired depth (that is, the depth of the tissue from which a biopsy sample is desired) in order to determine how far carriage 226, and therefore inner cannula driver 248 and outer cannula driver 250, may travel within housing 204 to throw depth stop 264 at the proximal end of guide 252.

Once the throw depth is selected and the device is fully cocked, the needle set may be injected for tissue sampling at a tissue entry site that the device operator has selected. To actuate the device, the operator moves actuator safety 238 out from its “safe” position preventing proximal displacement of actuator assembly 232 and then presses actuator 234, urging carriage arm ends 230 out from main catches 272. After releasing carriage arm ends 230 from main catches 272, actuator 234 is returned to its unactuated position by actuator repositioner 236. The operator's pressing action causes actuator 234 to be displaced proximally towards the carriage arm ends 230 in the main catches 272, and forces the carriage arm ends 230 out of the main catches 272. This release of the carriage arm ends 230 allows the release of potential energy in carriage drive assembly 256 (originally stored by the two-stage cocking of the device), which causes carriage 226, and therefore inner cannula driver 248 and outer cannula driver 250, to travel proximally until outer cannula driver 250 reaches the proximal end of needle set driver guide 252. Accordingly, the proximal end of needle set 202 travels away from the device and towards the tissue sampling site.

For embodiments in which a twisting motion is to be imparted to at least one of the cannulae, the corresponding cannula driver rotates during actuation of the device. For cylindrical cannula drivers, the driver may, by way of example, be part of a worm drive (wherein the driver includes a worm on its outer surface and the needle set driver guide or carriage assembly includes a worm gear, or vice versa) or, inter alia, be provided with a spiral groove and pin assembly. It is to be understood that various types of rotation-imparting means may be suitably used, the selection and configuration of which may depend on factors such as manufacturing costs, the degree of rotation desired, and the needle set travel distance over which such rotation is to be imparted.

In the embodiment illustrated in FIGS. 8 to 11, outer cannula driver 250 includes a helical groove 274 on its outer surface which slidingly engages pin 222 (shown in FIG. 2) of carriage assembly 224. Thus, when the proximal end of outer cannula driver 244 reaches the distal end of throw depth stop 264 and is prevented from travelling further proximally, carriage assembly 224 continues to move proximally for a distance no greater than the length of helical groove 274 and so causes outer cannula driver 244 to rotate in order to maintain engagement with pin 222. In various other embodiments, the helical groove might be provided on a carriage assembly and the pin might be provided on the surface of the cannula driver corresponding to the cannula which is to be twisted. In yet other embodiments, one of the helical groove and pin may be disposed on the cannula driver of interest, while the other of the pair may be disposed on a throw depth stop or needle set driver guide.

Once the device has been actuated and injected into tissue, and a biopsy sample captured by the needle set, the needle set may be withdrawn from the tissue site. To retrieve the biopsy sample from the needle set, the device may be cocked again to draw the needle set back. In embodiments including a rotating cannula driver, cocking the device after actuation causes the rotation of the cannula driver in a direction opposite to that which was caused by actuation. Post-actuation cocking also urges both cannula drivers distally. As inner and outer cannulae 242 and 244 are annularly arranged over the stylet portion of assembly 240 and as stylet assembly 240 remains stationary, the distal movement of cannula drivers 248 and 250 causes cannulae 242 and 244 to be drawn distally over the proximal end of stylet assembly 240, thereby exposing and releasing the biopsy sample.

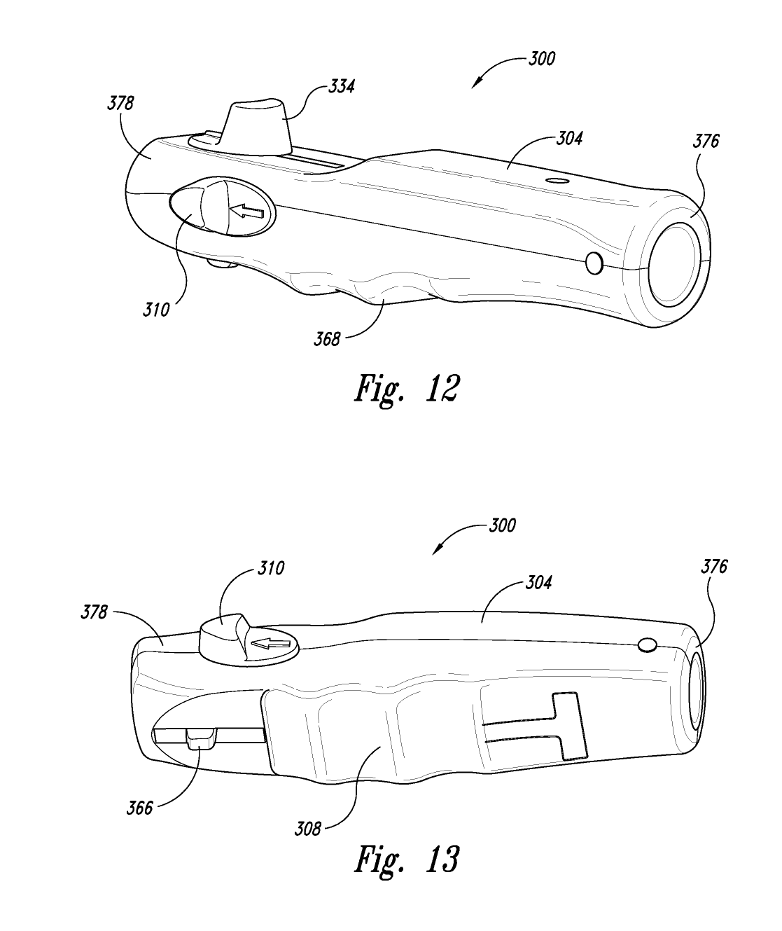

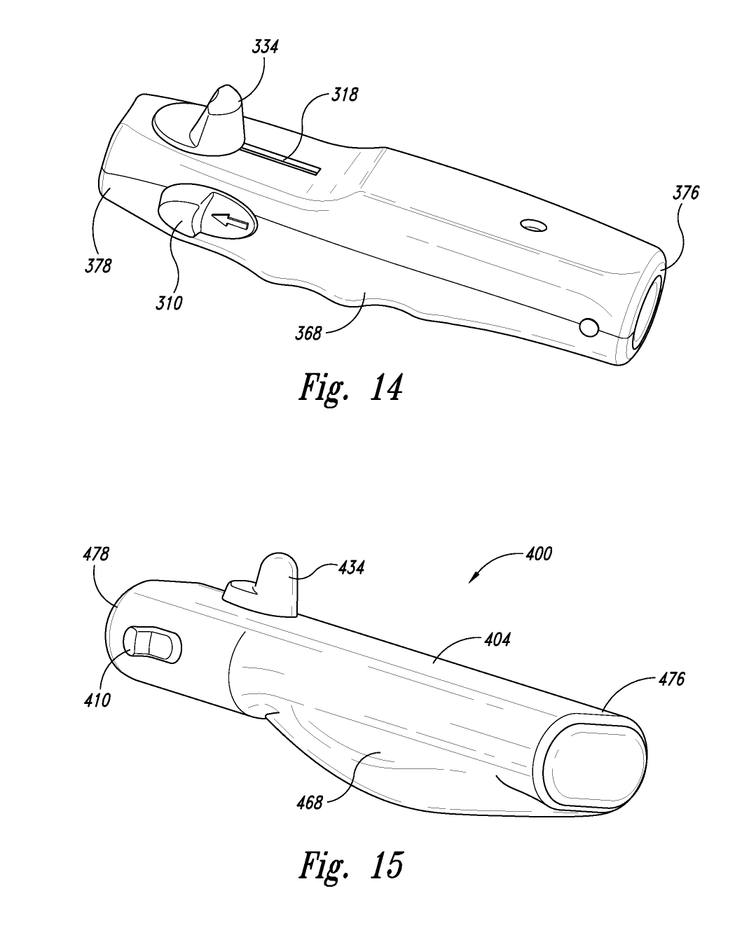

FIGS. 12 to 14 provide various views of a biopsy device handle exterior of an embodiment of the invention. Device handle 300 includes housing 304 having a distal end 378 and a proximal end 376, wherein the proximal end (through which a needle set would extend) is substantially circular. One side of housing 304 is provided with finger grips 368 to increase the security of the operator's hold on the device. Finger grips 368, actuator 310, and cocking button 334 are all arranged on three adjacent sides of the housing, with actuator 310 and cocking button 334 being disposed near distal end 378 of housing 304. Throw depth selector 366 may be disposed near distal end 378 either adjacent to finger grips 368 or on the remaining free side of housing 304. Assuming a right-handed operator of the device, the operator would align his or her fingers along finger grips 368 such that distal end 378 would be adjacent to the operator's wrist, and leaving the thumb free to slide cocking button 334 along housing aperture 318 to cock the device and push actuator 310 to fire it. (Of course, it is to be understood that the arrangement of parts illustrated in these figures could be reversed to accommodate a left-handed operator.)

FIG. 15 illustrates another biopsy device handle exterior of an embodiment of the invention. Device handle 400 includes housing 404 having a distal end 478 and a proximal end 476, wherein the proximal end (through which a needle set would extend) is substantially ovoid. One side of housing 404 is provided with a distension 468 of housing 404 to increase the comfort and security of the operator's hold on the device. Distension 468, actuator 410, and cocking button 434 are all arranged on three adjacent sides of the housing, with actuator 410 and cocking button 434 being disposed near distal end 478 of housing 304 while distension 468 is disposed from about the middle of the housing to about its proximal end. Throw depth selector (not shown) may be disposed near distal end 378 either adjacent to distension 468 or on the remaining free side of housing 404. Assuming a right-handed operator of the device, the operator would align his or her fingers along distension 68 such that distal end 478 would be adjacent to the operator's wrist, and leaving the thumb free to apply force to cocking button 434 to cock the device and push actuator 310 to fire it. (Of course, it is to be understood that the arrangement of parts illustrated in these figures could be reversed to accommodate a left-handed operator.)

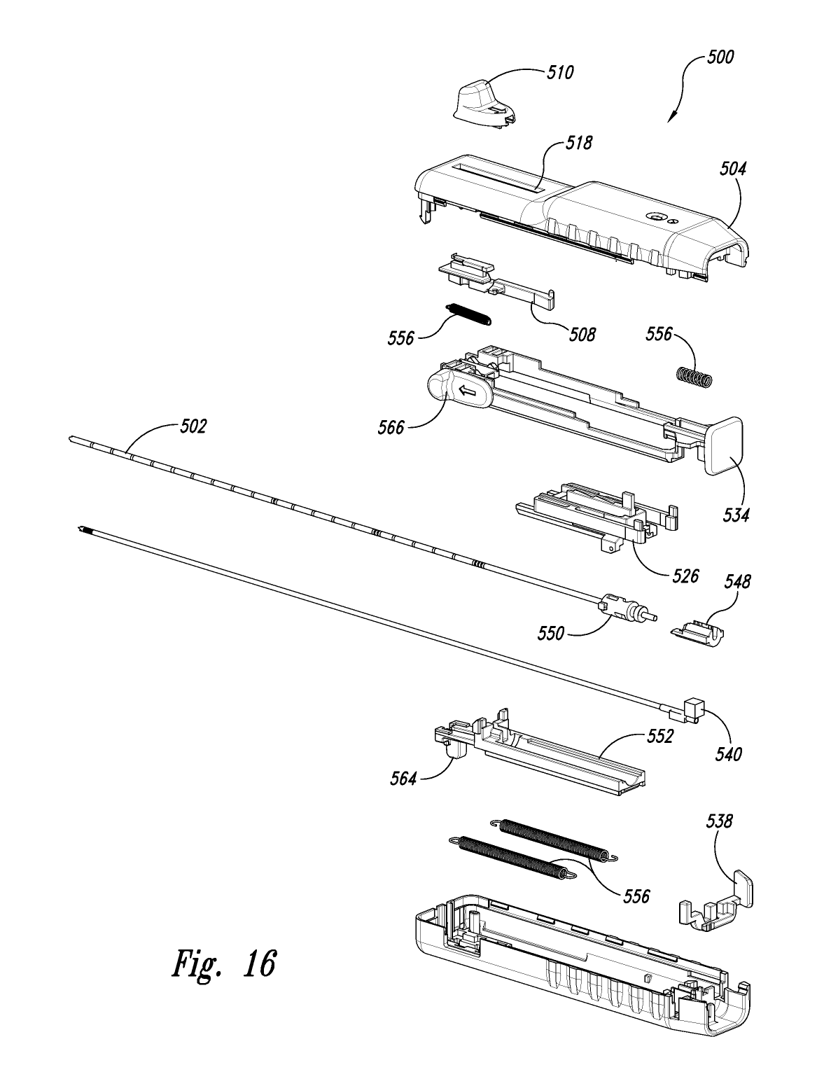

FIG. 16 provides an exploded view of another embodiment of the biopsy device handle of the present invention. In this embodiment, biopsy device handle 500 includes cocking button 510 which may be slidably disposed in aperture 518 of housing 504. Also present are cocking assembly 508 (which functions equivalently to features 108 and 208), actuator 534 (which functions equivalently to features 134 and 234) having an associated throw depth selector 566 (which in large part functions equivalently to 166 and 266), carriage 526 (which functions equivalently to features 126 and 226), stylet 540 (which functions equivalently to features 140 and 240), needle set driver guide 552 (which functions equivalently to features 152 and 252) associated with a throw depth stop 564 (which functions equivalently to 164 and 264), springs 556, and actuator safety 538 (which functions equivalently to 138 and 238).

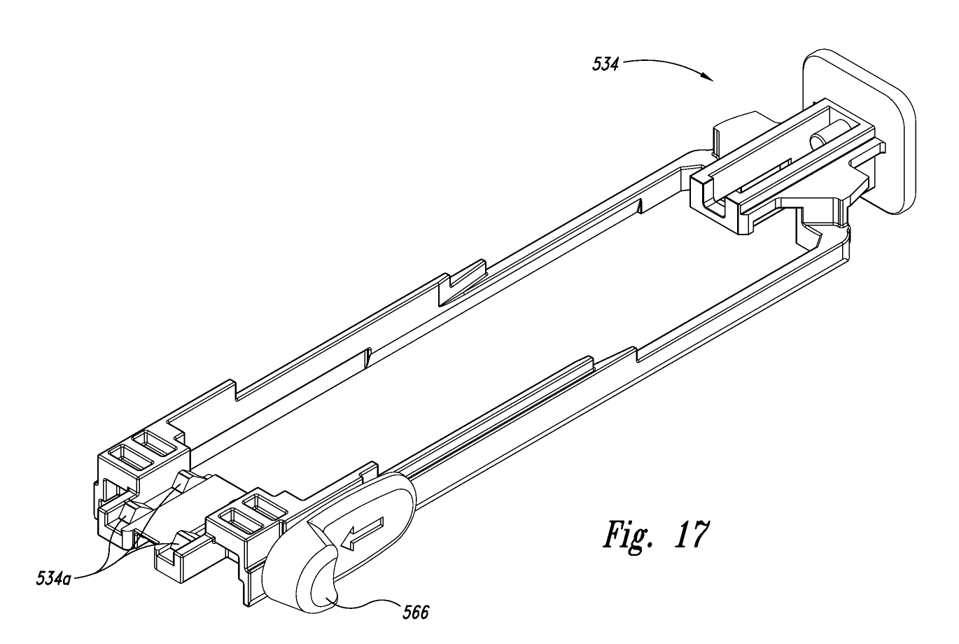

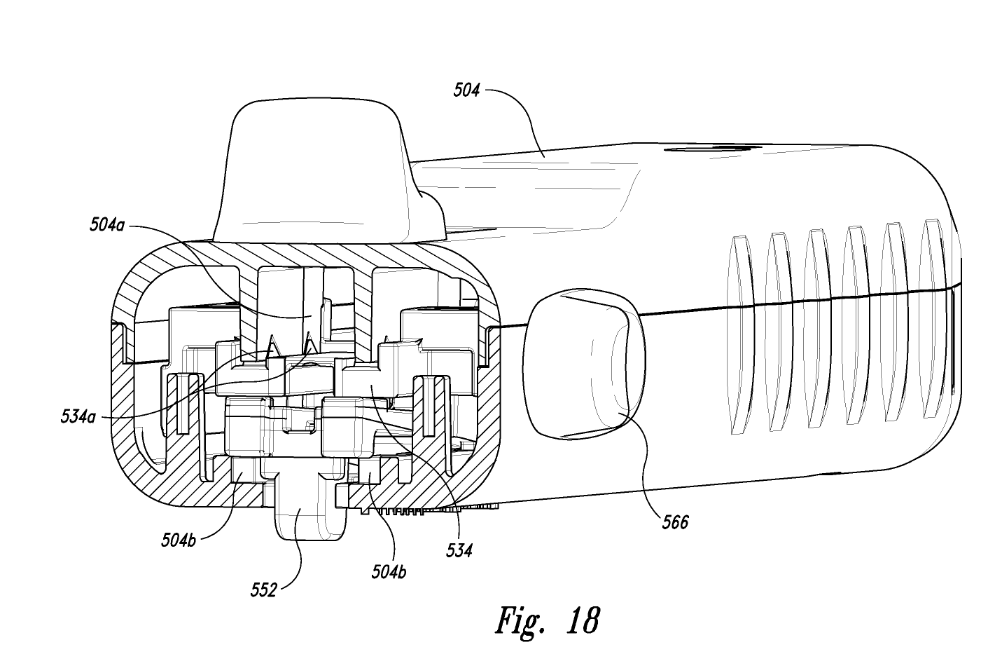

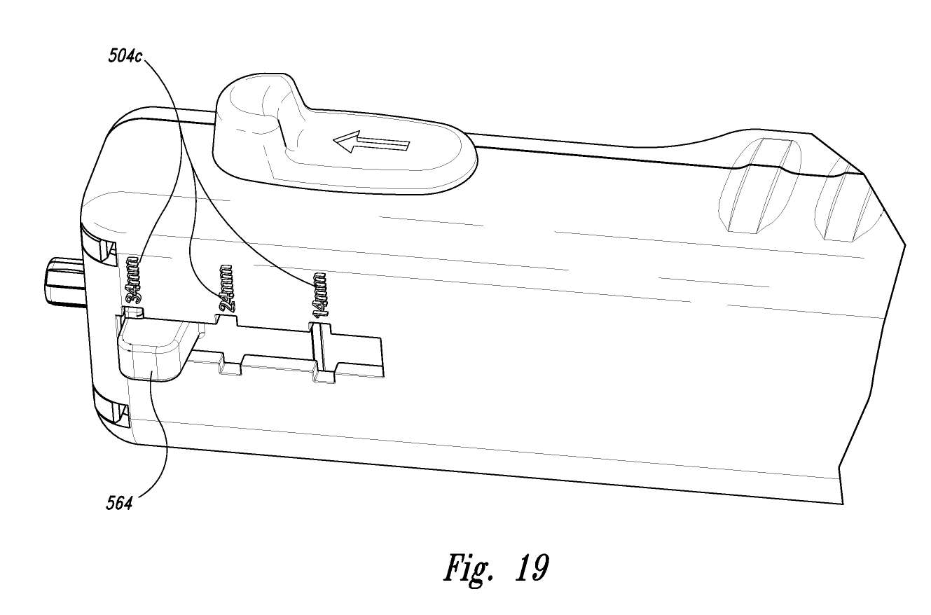

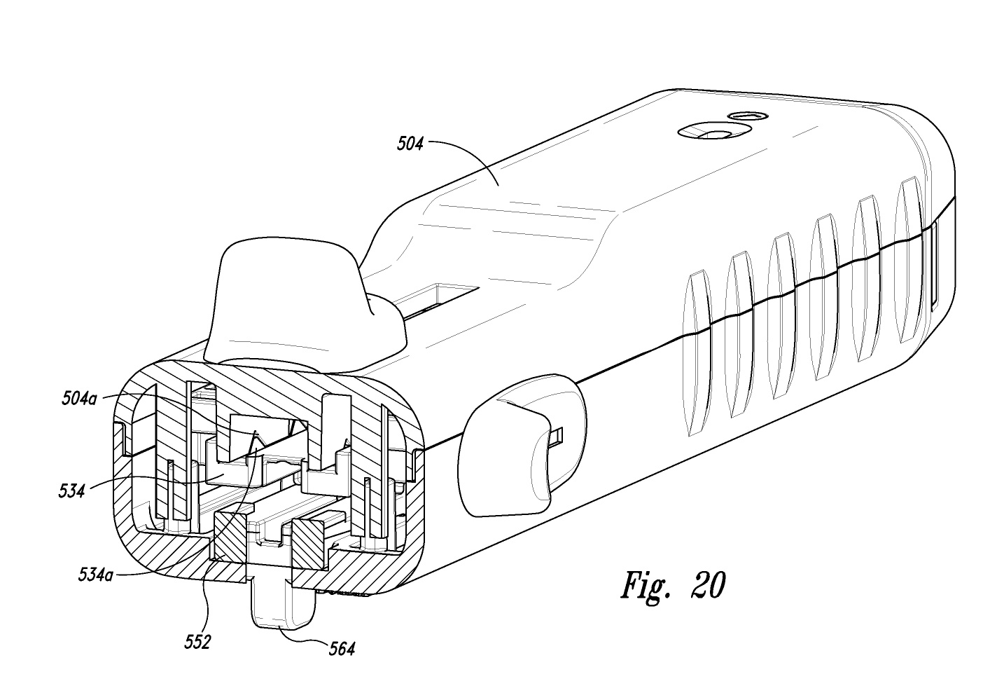

The actuator 534 of FIG. 16 is shown in expanded view in FIG. 17. The actuator 534 includes one half of a locking mechanism, shown as teeth 534a. The teeth engage complementarily shaped openings 504a in housing 504 to provide a locked position, where this locked position is held in place by contact between the actuator and the needle set driver guide 552. In turn, the needle set driver guide 552 is held in place as it rests against rails 504b of the housing 504. The rails 504b are located between the openings 504c, where the throw depth stop 564 can fit into any of the predetermined openings 504c as shown in FIG. 19, to thereby set the depth to which the needle will enter tissue to obtain a biopsy sample. When the throw depth stop 564 is located within an opening 504c, as shown in FIG. 19, the needle set driver guide will displace off the rails 504b and thereby be allowed to move away from the actuator 534, as shown in FIG. 20. With the needle set driver guide 552 no longer pressed against the actuator 534, the teeth 534a can disengage from the openings 504a, and the actuator 534 can move in a longitudinal direction. However, when the throw depth stop 564 is located between openings 504a, then the needle set driver guide 552 will rest on rails 504b and cause teeth 534a to be locked into openings 504a, thus precluding the biopsy device from firing.

Although the present invention has been shown and described in detail with regard to only a few exemplary embodiments of the invention, it should be understood by those skilled in the art that it is not intended to limit the invention to the specific embodiments disclosed. Various modifications, omissions, and additions may be made to the disclosed embodiments without materially departing from the novel teachings and advantages of the invention, particularly in light of the foregoing teachings. Accordingly, it is intended to cover all such modifications, omissions, additions, and equivalents as may be included within the spirit and scope of the invention as defined by the following claims.

What is claimed is:

1. A biopsy device handle for use with a biopsy device needle set, comprising a housing, a throw depth selection assembly, a cocking assembly, a carriage assembly, a needle set driver assembly, and an actuating assembly.

2. The biopsy device handle of claim 1, further comprising a stylet assembly.

3. The biopsy device handle of claim 1, wherein the needle set driver assembly comprises a plurality of cannula drivers for drivably mounting needle set cannulae.

4. The biopsy device handle of claim 3, wherein at least one cannula driver is rotatable relative to the housing.
